(19) United States
(12) Patent Application Publication
(10) Pub. No.: US 2021/0378491 A1
(43) Pub. Date: Dec. 9, 2021

(54) COLONOSCOPY PRESSURE RETENTION DEVICE

(71) Applicant: THE REGENTS OF THE UNIVERSITY OF CALIFORNIA, Oakland, CA (US)

(72) Inventors: Denise KALMAZ, Del Mar, CA (US); Brendan GONZALES, San Diego, CA (US); Andie LIN, Diamond Bar, CA (US); Di WU, Berkeley, CA (US); Namkyung HONG, San Diego, CA (US)

(21) Appl. No.: 16/646,122

(22) PCT Filed: Sep. 12, 2018
(86) PCT No.: PCT/US2018/050759

§ 371 Date: Mar. 10, 2020

Related U.S. Application Data
(60) Provisional application No. 62/557,664, filed on Sep. 12, 2017.

(51) Int. Cl. A61B 1/00; A61B 1/31

(52) U.S. Cl. CPC A61B 1/00131; A61B 1/31

(57) ABSTRACT

The pressure retention device includes a bottom flange configured to fit within a patient's intergluteal cleft in contact with an anal opening. A neck portion extending from the bottom flange has a co-axial channel therethrough to slidably receive an endoscope. An upper portion at an end of the neck portion has a pair of radially-extending resilient flaps that, when folded radially inward, form an insertion tip that relaxes to extend radially after insertion through the anal sphincter. An annular seal within the co-axial channel seals over the outer surface of the endoscope so that release of a pressurizing fluid through the anal sphincter is resisted. The components are molded as a single piece from a flexible silicone material.

### RELATED APPLICATIONS

This application claims the benefit of the priority of U.S. provisional Application No. 62/557,664, filed Sep. 12, 2017, which is incorporated herein by reference in its entirety.

### FIELD OF THE INVENTION

The present invention relates to a device for resisting leakage of distension fluid pressure during colonoscopy procedures.

### BACKGROUND OF THE INVENTION

Colorectal cancer (CRC) is the third most common cancer diagnosed in the United States. It is also the third leading cause of cancer-related death in women and the second leading cause in men. Fortunately, over the last two decades, the death rate from this cancer has declined significantly within the over-50 population in which it most commonly occurs. There are two primary reasons for this decrease: improvements in therapy and the increase in screening for and removal of colorectal polyps.

A colonoscopy is a endoscopic visual examination of the colon that is most commonly prescribed for adults over the age of fifty, when risks of colon cancer tend to increase. During the procedure, in which the patient is typically sedated, the patient lies on his or her side while the endoscopist inserts a half inch endoscope through the anus and into the colon. Most endoscopes have a camera and a light to allow visualization of the colon in real time as well as to record still and/or video images, along with one or more devices to resect polyps or suspicious growths. In order to clearly see within the colon, which is in a naturally collapsed state, air or water is used to inflate the organ. Based on a report by the American Society for Gastrointestinal Endoscopy in 2013, the mean pressure used to distend the colon during these procedures was 0.425 psi and a maximum of 1.102 psi. During the procedure, healthy anal sphincters normally create a tight seal around the scope to retain the pressure inside the colon. However, some patients, who through injury or disease have lost anal sphincter integrity, are unable to retain the pressure. This problem complicates the procedure, requiring the endoscopist to constantly pump more air or water into the colon to maintain the distention.

Currently, the standard practice for dealing with weak anal sphincters is for a technician to apply pressure using a towel to clamp the patient's buttocks, attempting to hold the anus closed around the scope so the air or water inside the colon will not leak. These techniques, however, are not always sufficient, extending the typical 30 minute procedure time to as much as an hour and a half.

One proposed solution to the stated problems is described by M. R. McEniry, et al. in J. Med. Devices 8(3), 030923 (Jul. 21, 2014). This device consists of a length of cylindrical tube 10 through which the endoscope may be inserted, as shown in FIG. 1. Annular balloons 12, 14 are concentrically mounted near opposite ends of the tube, one of which can be inflated internally 12, just inside the anal sphincter to hold the device in place, and a second external balloon 14 to create an outer seal. Air lines 16 and 18 provide means for inflating/deflating the two balloons. Additional proposed solutions are described by Simchony, et al., in U.S. Pat. Publ. 2015/0351617, which discloses an annular balloon positioned just inside the anus and a flange to contact the anus externally, and Binmoeller, et al., in U.S. Pat. Publ. 2016/0038007, which discloses a water-aided endoscopy process and a tool for use therewith.

Anal sphincter compromise is becoming increasingly common and can be secondary to increased age, childbirth, trauma, prior surgery, and anal intercourse. Appropriate solutions are needed to answer the growing need for colonoscopies among the aging population. An effective procedure relies on retention of a reasonable amount of air or water inside the patient's colon without excessive leakage. This retention must be achieved without exerting undue friction on the colonoscope, which can hinder maneuverability. Ideally, friction reduction should be prioritized over preventing leakage. The device must also stay in place and not slip out as a result of either the person's sphincters pushing it out or from forces applied during the procedure. Since the device is inserted into a body cavity, it cannot cause trauma to the anal canal and must be disposable for sanitary reasons. An additional advantageous feature is the ability to apply the device after the colonoscope has already been inserted into a person.

### BRIEF SUMMARY

In an exemplary embodiment, a device for retaining pressure during a colonoscopy procedure includes a base flange configured to sit against the patient's intergluteal cleft, a neck portion extending from the base flange and having a co-axial opening therethrough, an annular seal within the co-axial opening, and a pair of resilient flaps extending from an end of the neck portion opposite the bottom flange. The flaps are configured to fold down and conform to the colonoscope prior to insertion. After insertion, the flaps resile toward their original radial extension position, securing the device within the anal canal. The co-axial opening and annular seal are configured to fit closely over an outer surface of an endoscope so that when the flaps are inserted into a patient's rectum and the clamp portion is disposed substantially in alignment with the patient's anal canal, a pressurizing fluid introduced into the colon is substantially retained within the colon. All components of the pressure retention device are preferably molded from an elastomeric material that is biocompatible and flexible with a smooth surface finish, e.g., a medical-grade silicone.

In one embodiment, the device may include a slit running longitudinally along its length to allow it to be opened for positioning over the colonoscope after the scope has already been inserted into the patient's colon. A clip slides over the neck portion to seal the slit. The inventive design represents a significant advantage over prior art approaches that require the colonoscope to be completely withdrawn if the endoscopist discovers that the patient's anal sphincter integrity is insufficient to retain the needed pressure and that a pressure retention device is needed.

According to an exemplary embodiment, the pressure retention device (PRD) is designed to be positioned inside the anal canal at any time during the procedure, i.e., before or after the endoscope has been inserted into the patient. The PRD is made from medical grade polydimethylsiloxane (PDMS), a commonly used silicone, which is safe for colonic surface contact. Typical properties of such silicones include a Shore A hardness durometer in a range of 30-80 HA and a tensile strength in the range of 1.5 to 9 MPa (218-1305 psi). The PRD includes an inner seal that seals against the colonoscope, tested to withstand 1.3 psi of water pressure with less than 20 mL of leakage per minute, to retain pressure between the endoscope and the device with less than 1 Newton of friction after lubrication. The device has a slit down the middle so that it can be opened and placed around the endoscope at any time during the procedure and the addition of a rigid clip holds the device together. The outer form of the device is designed such that the neck of the PRD will sit against the sphincter muscles creating another seal between the device and the anal canal. The device includes two conical flaps that flex forward for insertion and spring backward to hold the position of the device throughout the rest of the procedure.

In one aspect of the invention, a pressure retention device includes a bottom flange configured to fit within a patient's intergluteal cleft in contact with an anal opening, the bottom flange have a central flange opening; a neck portion extending from the bottom flange, the neck portion having a co-axial channel therethrough aligned with the central flange opening, the co-axial channel having an annular seal therein, the neck portion configured to extend from an exterior of the anal opening through an anal sphincter; and an upper portion disposed at an end of the neck portion opposite the bottom flange, the upper portion having a central upper opening aligned with the co-axial channel and pair of resilient flaps extending radially therefrom, each flap having a partial channel so that, when the flaps are folded radially inward, the partial channels define an extension of the co-axial channel and the flaps form an insertion tip; wherein the co-axial channel and the annular seal are configured to fit closely over an outer surface of an endoscope so that, when the flaps are inserted into the patient's anal opening and the neck portion is disposed substantially in alignment with the anal sphincter, the flaps at least partially relax to radially extend from the upper portion. The flaps may be connected to the upper portion by a grooved hinge, wherein the flaps fold along the hinge upon application of pressure to an outer portion of each flap. In a preferred embodiment, the bottom flange, neck portion, and upper portion are formed as a single piece of a flexible, biocompatible material. A continuous longitudinal slit may be formed through a side of each of the bottom flange, neck portion, and the upper portion to provide radial access to the co-axial channel for positioning of the device over the endoscope. A semi-cylindrical clip is configured to slide over the neck portion to seal the slit after the device is positioned over the endoscope. In some embodiments, the flexible, biocompatible material is a medical-grade silicone and may be polydimethylsiloxane (PDMS). The flexible, biocompatible material may have a Shore A hardness within a range of 40 HA to 60 HA. The annular seal may be a rib that extends radially inward, angled toward the upper portion. The bottom flange preferably has an oval shape with a long axis aligned with the patient's intergluteal cleft. A ridged topography aligned with the long axis of the oval shape may be provided to ergonomically conform to the patient's intergluteal cleft. The flaps may have a tapered lower surface so that the insertion tip has a generally conical shape when the flaps are folded inward.

In another aspect of the invention, the pressure retention device includes a neck portion having a co-axial channel therethrough dimensioned to slidably receive an endoscope; an annular seal extending radially inward within the co-axial channel to contact an outer surface of the endoscope; a bottom flange disposed at a first end of the neck portion, the bottom flange configured to fit within a patient's intergluteal cleft in contact with an anal opening; and an upper portion disposed at a second end of the neck portion, the upper portion having a pair of radially-extending resilient flaps that, when folded radially inward, form an insertion tip that resiles to extend radially after insertion through the anal opening, wherein each flap has a tapered lower surface so that the insertion tip has a generally conical shape when the flaps are folded inward; wherein the annular seal generates a pressure seal to resist release of a colon pressurizing fluid through the anal opening. The flaps may be connected to the upper portion by a grooved hinge, wherein the flaps fold along the hinge upon application of pressure to an outer portion of each flap. In a preferred embodiment, the neck portion, bottom flange, and upper portion are molded from a single piece of a flexible, biocompatible material, which may be polydimethylsiloxane (PDMS). The flexible, biocompatible material may have a Shore A hardness within a range of 40 HA to 60 HA. A continuous longitudinal slit may be formed through a side of each of the neck portion, bottom flange, and upper portion for accessing the co-axial channel radially for positioning of the device onto the endoscope. A semi-cylindrical clip may be provided to slide over the neck portion to seal the slit after the device is positioned over the endoscope. The annular seal preferably comprises a rib extending radially inward and angled toward the upper portion. The bottom flange preferably has an oval shape with a long axis aligned with the patient's intergluteal cleft. A ridged topography aligned with the long axis of the oval shape may be provided to ergonomically conform to the patient's intergluteal cleft.

### BRIEF DESCRIPTION OF THE DRAWINGS

FIG. 1 is a perspective view of a prior art sealing device.

FIG. 2 is a front perspective view of an embodiment of the invention.

FIGS. 3A-3D show the inner seal according to an embodiment of the invention, where FIG. 3A is a cross-sectional view taken along line A-A of FIG. 2; FIG. 3B is a cross-sectional view taken along line B-B of FIG. 3A; FIG. 3C is a partially cut away perspective view of the inner seal; and FIG. 3D is a cross-sectional view of an alternative rounded seal configuration.

FIG. 4 is a perspective view of the top flap assembly according to an embodiment of the device.

FIG. 5 is a perspective view of the base flange of an embodiment of the invention.

FIG. 6 is a front perspective view of the device and clip according to an embodiment of the invention.

FIG. 7 is a cross-sectional view through the neck portion of the device with the clip in place.

FIG. 8 is a rear perspective view of the device showing the openable split.

FIG. 9 is a perspective view showing an embodiment of the device as used with a colonoscope during insertion.

FIGS. 10A-10C are photographs of the four-piece mold for fabricating a prototype embodiment of the inventive device, where FIG. 10A shows the disassembled pieces, FIG. 10B shows three pieces assembled, and FIG. 10C shows the assembled mold.

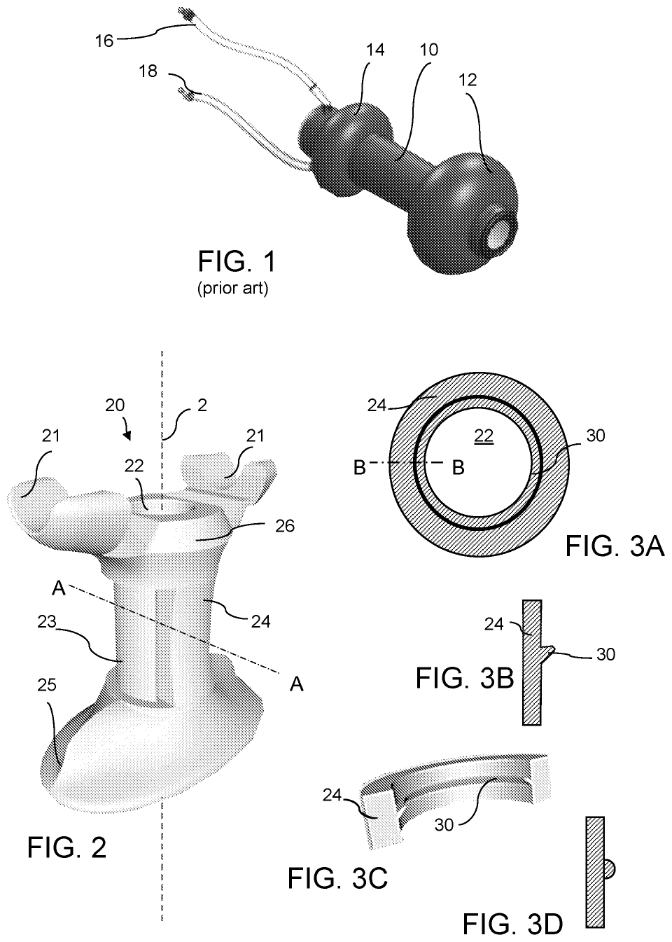

### DETAILED DESCRIPTION OF EXEMPLARY EMBODIMENTS

The written description of embodiments of the invention refer to directions, e.g., front, rear, upper, lower, etc., which are provided to facilitate understanding relative to the drawings. Such directional references are not intended to be limiting with respect to the use or positioning of the disclosed device.

This colonoscopy pressure retention device (PRD) of the present invention is designed to assist practitioners performing a colonoscopy procedure by retaining the pressurizing fluid within the colon, in effect acting as an artificial sphincter. The device may be configured to be able to be applied after the colonoscope has already been inserted into a patient. The PRD has four basic components: a bottom flange 25 and an upper flap portion 26 disposed at opposite sides of a neck portion 24. The PRD has a co-axial bore or channel 22 extending therethrough configured to fit over the outer surface of a colonoscope. The surface of channel 22 forms an inner seal against the outer surface of the colonoscope. A longitudinal slit 34 allows the PRD to be opened to slide over the colonoscope. A tubular clip 60 formed from a semi-rigid materials fits over the outer surface of the PRD to produce an appropriate seal.

FIG. 2 illustrates an embodiment of the PRD 20 positioned relative to a central longitudinal axis 2, which is provided for reference. The upper portion 26 of the device includes two diametrically disposed resilient contoured flaps 21 that can be folded inward toward central axis 2 under manual pressure to act as an insertion tip for placement of the device. After insertion into the patient's anus, the flaps 21 are released, allowing them to relax back toward their original position, thus acting as a partial stop to retain the device within the patient's anal sphincter. The neck portion 24 has a generally cylindrical shape that may be slightly radially flared toward the ends of the neck portion where the neck portion connects to the upper portion 26 and the base flange 25, respectively. Base flange 25 is generally oval in shape with ridges formed in its upper surface parallel to the long axis of the oval to ergonomically sit against the intergluteal cleft, preventing movement of the PRD, and sealing against the patient's anus. The long axis of the oval is also parallel to the flap extensions. A central channel 22 extends the length of the device to provide slidable insertion of a colonoscope. The entire device is preferably molded as a single piece unit from a flexible biocompatible material, such as a medical-grade silicone. In a preferred embodiment, the material is polydimethylsiloxane (PDMS).

FIGS. 3A-3C illustrate the inner seal 30 that is formed on the interior of the neck portion 24 of the PRD. Inner seal 30 extends radially inward into central channel 22 to form a slidable seal against the outer surface of the colonoscope. A balancing is required to obtain an acceptable pressure seal without unduly hindering the ability to maneuver the scope as the result of friction. From the perspective of the practitioner, the priority is to reduce friction over the seal, so the diameter of the seal was selected to be slightly smaller than the smallest diameter of the scope, noting that the scope itself may not have a uniform diameter. While a standard rounded O-ring configuration may be used with a semi-circular cross-section extending from the inner wall of the neck portion, as shown in FIG. 3D, a preferred seal configuration is one with a triangular cross-section as shown in FIG. 3B, in which the annular ribbed structure is angled toward the upper portion 26 of the device, i.e., in the direction of the patient's colon. This angled rib configuration makes it easier to insert and guide the scope into the patient's colon with minimal frictional binding while generating a good seal around the scope. The inwardly angled rib 30 will tend to generate more friction upon withdrawal of the scope, however, at this point in the procedure, the endoscopist will generally not be as concerned about fine maneuverability.

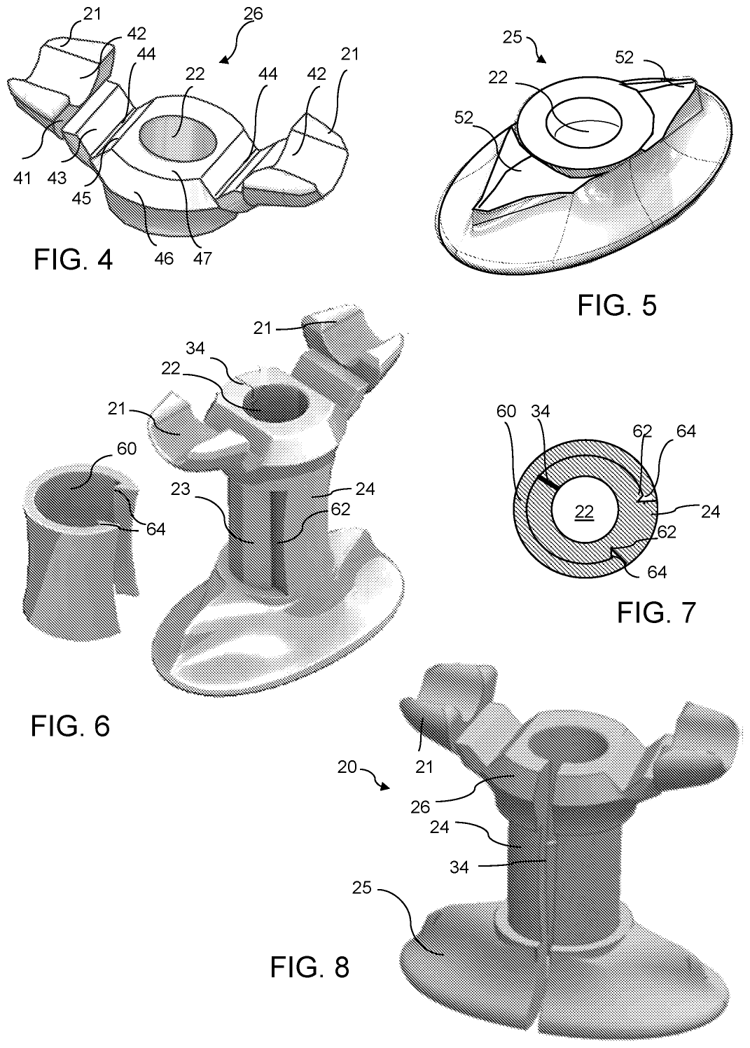

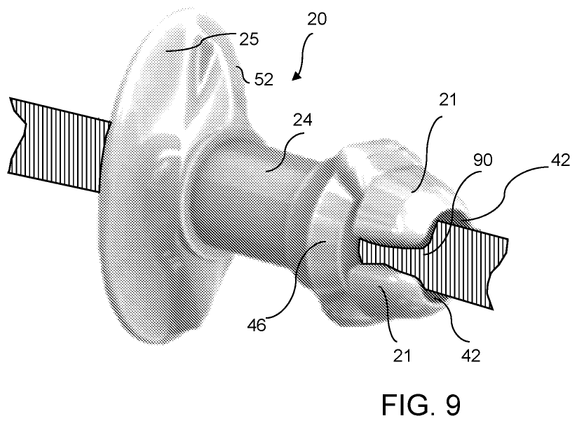

The flaps 21 in upper portion 26 are configured to hold the air retention device in place against the anal canal of a wide range of patients with different sized sphincters. The flaps provide resistance to movement of the PRD while the scope is being inserted or withdrawn. FIG. 4 illustrates details of the upper portion 26 of the PRD 20. The two flaps 21 extend in opposite directions radially away from central channel 22. Each flap 21 has rounded outer edges with a semicircular channel 42 bisecting the flap. When the flaps are folded inward, the channels 42 combine to form an extension of central channel 22 through which the colonoscope 90 passes, as shown in FIG. 9. The rounded outer edges of the flaps combine with curved beveled surface 46 to define a smooth semi-conical structure that acts as a tapered insertion tip. Each flap 21 is connected to the side of upper portion 26 by a grooved hinge structure 44 that allows the flap to fold over to sit flat on the top plane of upper portion 26 to define the conical insertion tip. Specifically, folding along the groove of hinge structure 44 by pressing upward and inward on the flap causes flat surfaces 43 and 45 come into contact with each other, in turn causing flat surface 41 of flap 21 to contact the top surface 47 of upper portion 26. The uniform load distribution across these flat surfaces enhances the strength and stability of the insertion tip shown in FIG. 9.

FIG. 5 illustrates the base flange 25 of the PRD. The flange 25 is ergonomically configured to fit closely within the patient's intergluteal cleft. The long axis of the oval shape is aligned with the cleft as is a tapered ridge 52 that is formed in the flange's upper surface to provide a topography that generally conforms to the cleft shape. The flange is formed with smooth surfaces to follow the natural contours of the patient's anal area, producing a secure seal against the exterior anal surface once the flaps 21 of the upper portion have been inserted through the anal sphincters.

Referring to FIGS. 6-8, in some embodiments, a slit 34 may be formed along the length of the device, i.e., through the upper portion 26, neck portion 24, and base flange 25, to allow the device to be manually spread apart to fit over a colonoscope from the side, allowing PRD 20 to be positioned after the colonoscope has already been inserted into the patient's colon. A shallow recessed area 23 is formed within the neck portion 24 to receive a semi-cylindrical clip 60 to form a surface that will be in direct contact with the patient's anal sphincter. Clip 60, which is semi-rigid to provide sufficient flexibility expand and slide over the neck portion 24 from the side, includes ridges 64 that protrude inward to capture corresponding channels 62 formed at the edges of recessed section 23 to secure the clip. Upon assembly, the outer surface of clip 60 should be flush with the outer surface of the neck portion 24 to define a smooth, substantially continuous surface. The semi-rigid polymer material, e.g., resin, used to form clip 60 lends it rigidity to the assembled PRD, thus facilitating handling of the device for insertion and removal.

The material of which the PRD is formed is biocompatible with a soft durometer and smooth surface to minimize trauma to the patient. For purposes of the present description, “biocompatible” refers to the ability of a material to perform with an appropriate host response in a specific situation. The type of material required depends on the function of the device, the tissue being contacted, as well as the duration of its use. Typical colonoscopies take around 30 minutes, however, some can last as long as 90 minutes, so the device could be in contact with the patient's anus for up to 2 hours.

The PRD material is ideally flexible and tear resistant. Typical properties of appropriate elastomers and silicones include a Shore A hardness durometer in a range of around 25 to 80 HA and a tensile strength in the range of 1.5 to 9 MPa (218-1305 psi). The PRD includes an inner seal that seals against the colonoscope, tested to withstand 1.3 psi of water pressure with less than 20 mL of leakage per minute, to retain pressure between the endoscope and the device with less than 1 Newton of friction after lubrication. In early prototyping and for some embodiments, the PRD was fabricated using 3-D printing of materials with flexible, rubber-like qualities. Examples of such materials include Tango Black™ (Shore A hardness of 26-28 HA), which was used alone or mixed with a more rigid polymer (VeloClear™) to produce a range of hardnesses. Both products are available from Stratasys, Ltd. (Eden Prairie, Minn.). In other embodiments, the material used was a silicone rubber, which is widely commercially-available. Examples of appropriate materials include the ECOFLEX™ line of silicone rubbers from Smooth-On, Inc. (Macungie, Pa.) and the SYLGARD™ silicone elastomers from The Dow Chemical Company. For prototyping, SYLGARD™ 184 polydimethylsiloxane (PDMS), with a manufacturer-specified Shore A hardness of about 43 HA, was selected for use in molding the PRD. For patient use, a medical grade PDMS silicone such as products commercially-available from Shin-Etsu Silicones of America, Inc., e.g., SV-46020U (Shore A hardness of around 59 to 65 HA) or similar, may be used. Selection of appropriate materials from other sources will be within the level of those of skill in the art.

At room temperature, PDMS behaves like a thick liquid. When mixed with a curing agent, the polymer hardens after several hours into a clear, soft, deformable, and hydrophobic material. The curing process of PDMS is clean and produces no by-products and thus does not lose volume, allowing it to be molded at high precision. The suggested ratio from the manufacturer is 10:1 by weight elastomer base to curing agent, however, the properties of the PDMS can be manipulated by varying this ratio. The curing process of the PDMS can be accelerated by heating the material, however, heating may affect the hardness and structural integrity of the material. During molding, air bubbles that may form during the mixing process should be eliminated. This can be done by placing the mixture into a vacuum chamber to expel the air bubbles for a smoother bubble free mixture.

The above-described liquid molding process was used to fabricate prototypes for testing. As will be readily apparent to those of skill in the art, other fabrication techniques may be employed to enable cost efficient and high volume production. For example, injection molding, compression molding, or other silicone manufacturing techniques are well-known in industry

The cured PDMS material is flexible and resistant to tearing under the normal usage loads that would be involved during use of the inventive device. However, the presence of air pockets in the material could compromise the integrity of the solid and significantly reduce its ability to resist tearing. Thus, it is important that all impurities, particularly air pockets, are eliminated, especially near the upper flap, since it will experience the greatest stresses.

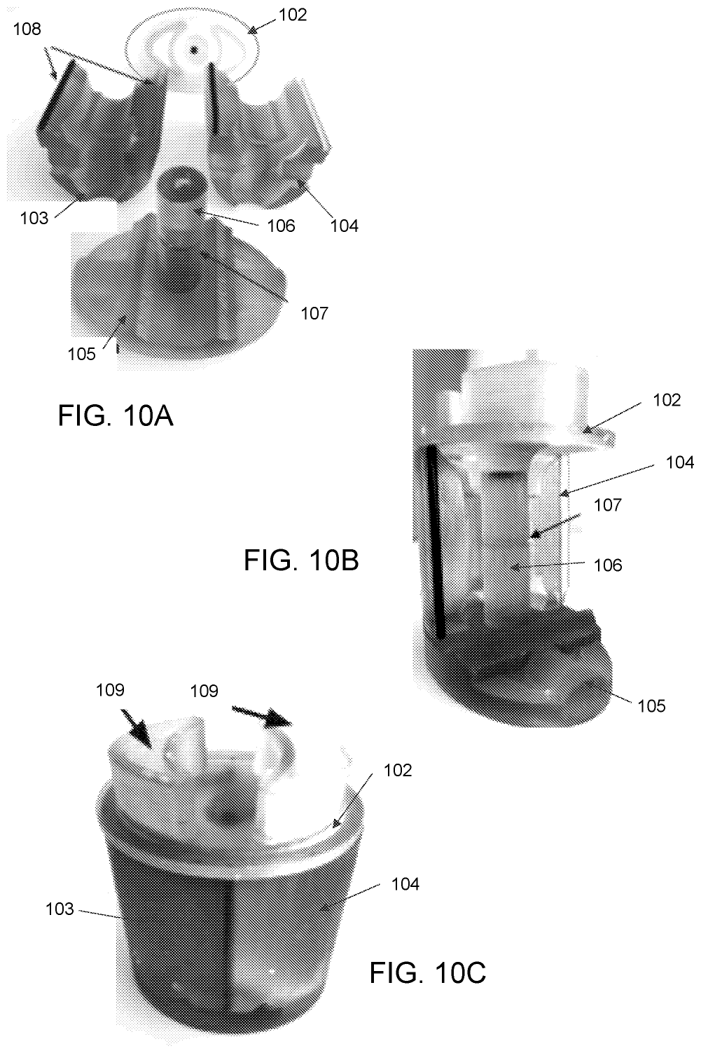

In the exemplary embodiment, the PRD (excluding clip 60) is fabricated as a single piece using a four piece mold. FIGS. 10A-10C are photographs of a prototype mold, which includes a top section 102, mid-left section 103, mid-right section 104 and bottom section 105. The bottom section 105 includes a center tube 106 that defines the central channel 22 and has a groove 107 for forming seal rib 30. Key slots 108 are provided in maintain correct relative positions of the mold parts and to prevent the PDMS from leaking out. The PDMS liquid is introduced into the assembled mold through inlets 109 shown in FIG. 10C.

The PDMS was prepared as suggested by the manufacturer, with a ratio of 10:1; base to curing agent. The mixture is stirred for about 5 minutes to completely mix. To minimize air bubbles, the container is placed into a vacuum chamber, which is gradually reduced to about −14 psi. Keeping the chamber at this pressure will cause the air bubbles to rise out of the PDMS. This pressure is maintained for 10 to 20 minutes, or until the PDMS begins overflowing out of the cup. The chamber then is gradually increased to about −7 psi to pop the air bubbles. The sequence of depressurizing and pressurizing may be repeated until the solution appears to be free of air bubbles.

After coating the interior walls of the mold with an appropriate mold release solution, the PDMS is slowly poured into the mold to minimize the chance of introducing air bubbles into the solution. Once the mold is filled, it is placed in the vacuum chamber and held until no further air bubbles surface, prior to capping the top of the mold. The vacuum step may be repeated as need to ensure that all air bubbles has been removed, after which the mold is capped. The lowest Shore A durometer can be achieved by allowing the PDMS to cure at room temperature for 48 hours. Heating of the PDMS-filled mold can accelerate the cure time, but introduces trade-offs in terms of hardness and brittleness. For testing, the mold was placed in an oven at 60 degrees Celsius for one hour and then allowed to sit for another hour. After curing and removal from the mold, the longitudinal slit 34 is created by cutting with a blade. For prototype construction, clip 60 was separately 3-D printed using a semi-rigid resin-like material with the Stratasys 3D printer. The clip should be able to flex enough to open and slide transversely over the neck portion, while being rigid enough to hold the device together. For commercial production, clip 30 may be injection molded or fabricated using known techniques for formation of semi-rigid medical-grade plastics or polymers.

The following examples describe testing and evaluation of the characteristics and performance of an embodiment of the inventive PRD:

### Example 1: Testing: Water Pressure

The purpose of water pressure testing was to determine how well a seal created by the inner seal design performed under a designated pressure. The maximum pressure used in colonoscopies is 1.102 psi. Therefore, to account for any error in pressure testing, and to ensure that the seals can handle the maximum anticipated pressure, the seals were tested against 1.301 psi, about 30% higher than the standard maximum pressure used during the procedures. The main goal of the PRD is to retain the pressure inside the colon by reducing or eliminating leaking from a weak sphincter. Thus, it was critical to find the right design and dimension of the seal against pressure. The water pressure test was performed by having the device attached to one end of a ten feet long tube filled with water, while raising the other end of the tube to designated heights to induce a specified amount of static pressure. The leakage within one minute was measured using a 200 ml beaker.

The results are based on the leakage condition of the device against about 0.9 m (˜3 ft) of water, which is approximately equal to the maximum pressure inside the patient's colon during a colonoscopy. As shown in Table 1, a smaller diameter inner seal tends to have less leakage per minute.

TABLE 1

| | |
|---|---|
| Seal Type | Pressure at 1.3 psi (water pressure at 0.9 m) |

| | | |
|---|---|---|
| Rounded | 1.22 cm/0.48 in. | >200 ml/min |
| (e.g., FIG. 3D) | 1.24 cm/0.49 in. | >200 ml/min |
| | 1.27 cm/0.50 in. | >200 ml/min |
| | 1.3 cm/0.51 in. | >200 ml/min |
| Angled, ribbed | 1.22 cm/0.48 in. | 20 ± 3 ml/min |
| (e.g., FIG. 3B) | 1.24 cm/0.49 in. | 80 ± 3 ml/min |
| | 1.27 cm/0.50 in. | 200 ± 10 ml/min |
| | 1.3 cm/0.51 in. | >200 ml/min |

During testing, the leakage per minute from four conventional round (semi-circular) seals ranging from 1.22 cm (0.48 in.) to 1.3 cm (0.51 in.) overflowed the beaker, indicating that a regular round seal would not sufficiently retain the pressure during the procedure. In contrast, as one decreases the inner diameter of angled rib knife-edge seal, i.e., seal 30 described above, the leakage per minute was significantly reduced. The ribbed seal with an inner diameter of 1.2 cm (0.48 in.) reduced the leakage per minute down to 20 ml per minute, which falls into an acceptable range.

### Example 2: Testing: Friction

Friction testing was conducted to evaluate the friction between the colonoscope and the inner seal. The test was performed using a spring gauge attached to a colonoscope analog, which was a 1.3 cm (0.51 in.) aluminum rod with electrical tape wrapped around it. The gauge was pulled at a constant force away from the air retention device, simulating both insertion and withdrawal conditions. The friction test was designed to quantify the friction force of various possible seal designs and dimensions, to allow balancing of friction versus seal quality.

TABLE 2

| | | Diameter | | | |
|---|---|---|---|---|---|
| | | 1.22 cm/ | 1.24 cm/ | 1.27 cm/ | 1.3 cm/ |
| Seal type | Direction | 0.48 in. | 0.49 in. | 0.50 in. | 0.51 in. |
| Angled, | Insertion | 0.5 ± 0.05 | 0.3 ± 0.05 | 0.2 ± 0.05 | 0.1 ± 0.05 |
| ribbed | (N) | | | | |
| (e.g., | Withdrawal | 0.9 ± 0.05 | 0.7 ± 0.05 | 0.6 ± 0.05 | 0.5 ± 0.05 |
| FIG. 3B) | (N) | | | | |
| Rounded | Insertion | 2 ± 0.05 | 1.7 ± 0.05 | 1.4 ± 0.05 | 0.9 ± 0.05 |
| (e.g., | (N) | | | | |
| FIG. 3D) | Withdrawal | 2 ± 0.05 | 1.7 ± 0.05 | 1.4 ± 0.05 | 0.9 ± 0.05 |
| | (N) | | | | |

The friction differences between different designs primarily resulted from differences in the dimensions of the inner seal. A smaller diameter of inner seal tends to have more friction, but at the same time it would improve the seal around the colonoscope. The goal was to have the friction below 1 Newton. A regular rounded inner seal with a diameter of 1.3 cm (0.51 in.) generated friction of 0.9N, thus achieving the goal of friction less than 1N. The knife-edge (angled ribbed) seal with a smaller diameter of 1.22 cm (0.48 in.) generated friction below 1N while producing a better seal. Based on the results from water pressure testing, the 0.48 in knife-edge seal was chosen over the 1.3 cm regular (rounded) seal due to its superior performance against pressure. The rod (colonoscope analog) in the friction test had a diameter of 1.3 cm, slightly larger than that of a typical colonoscope, which is about 1.27 cm (0.5 in.). The difference has been taken into consideration when making the design decision on the inner seal diameter, which was selected to be 0.47 in.

### Example 3: Testing: Durometer

The purpose of the durometer test was to determine the softness of the material used in fabrication. The air retention device is required to avoid causing trauma on patient. This test was performed by pushing the Shore A Durometer gauge against the material being tested until the reading on the gauge becomes stable.

As noted above, early prototypes were generated using 3-D printed materials. The 3-D printing materials were replaced with molded PDMS to reduce cost, improve practical manufacturability, and mimic materials that would ultimately be used for an actual medical grade device. Testing revealed that the durometer of the PDMS-based PRD was much higher than that of the 3-D printed Tango Black™ version: 47 HA versus 21.5 HA. The hardness differential arises from combination of the manufacturer's designed durometer (43 HA) and heating the mold to accelerate the cure time. A durometer of 21.5 HA was the original desired goal, however, testing on both swine and cadavers (described below) indicated that a device with a higher durometer was preferred for rigidity and ease of manipulation. A subsequent target value for durometer hardness was determined to be somewhat higher, in a range of around 40 to 65 HA. As previously noted, the Shin-Etsu silicone product selected for the device for patient use, SV-46020U, has a Shore A hardness of 59 HA to 64 HA, which falls within the target range.

### Example 4: Pig Test

Porcine models are commonly used for training and experimental testing of endoscopic and laparoscopic procedures. While the swine colon is quite different from that of the human, the focus of the present testing was on the effectiveness of the anal seal for retaining the pressurizing fluid (air or water), so the difference in colon configuration was not considered significant. Preparation of the animal for the procedure utilized conventional procedures, including bowel evacuation and sedation. Appropriate procedures for prep and sedation are described in the literature. See, for example, Swine in the Laboratory—Surgery, Anesthesia, Imaging, and Experimental Techniques, M. M. Swindle, editor, 2^{nd} Edition, 2007, CRC Press, Boca Raton, Fla.

An anesthetized four-foot swine positioned on its side was used to test three prototypes of the PRD made from different materials: (A) a soft PDMS (35 HA), (B) Tango Black™/VeroClear™ blend (3-D printed; 40 HA), and (C) a stiff PDMS (47 HA). The colonoscope was inserted and the distending fluid applied to expand the colon for visualization. Separate tests were performed using water and air. The pig's anus was loose, so there was already some leakage before applying the device. The looseness facilitated application of the device over the already-inserted scope.

Results: Prototype A, the soft PDMS, sealed well and produced negligible friction against insertion and retraction of the scope. The softness of the material was problematic because the device was unable to hold its position. Prototype B sealed well, generated negligible friction, and held its position during movement of the scope. Prototype C did not seal well, generated some friction, but held its position. Inspection of the seal revealed a defect that appeared to have occurred during the molding process. Nonetheless, the test was considered a success in its confirmation that the device would hold its position during use. The overall results for Prototypes B and C were particularly encouraging given that the device was able to hold its position in spite of continuous efforts by the sedated pig to expel the device and scope.

The primary challenge during testing was the PRD slipping out of the rectum during the procedure. This appears to have been caused by thicker sections of the scope (near the camera) getting caught on the inner seal. It is believed that this would not be an insurmountable problem because the thicker sections of the scope usually only come into contact with the inner seal at the beginning or end of the procedure, so extra attention could be given at these points to support the PRD, or the PRD could be placed after the camera has been inserted past the anus at the beginning of the procedure. In addition, at least some slippage of the device could be attributed to the anatomical differences of the pig's anal canal since, unlike humans, there are no buttocks to hold the device in place. The soft PDMS (prototype A) had problems remaining stationary on all sections of the scope because it was too flimsy. Prototype B (Tango Black™ blend), with a durometer hardness of 40 HA, proved to be sufficiently stiff to hold its position.

### Example 5: Human Cadaver Test

Prototypes A and B were tested in the human cadaver. Difficulty in handling the cadaver made it impractical to position it on its side for the procedure, so the body was placed flat on its back on a table prior to insertion of the scope. The PRD was placed over the scope.

Results: Prototype A, the soft PDMS, sealed well in both air and water tests, produced negligible friction against insertion and retraction of the scope, and held its position, although it is possible that the position of the body helped keep the PRD in place. Prototype B sealed well for both fluids, generated negligible friction, and held its position during movement of the scope.

The results of the cadaver test were similar to those of the pig test. As expected, the cadaver had no anal tone, but the device remained in place without problems. The position of the body caused the device to rest on the table, which may have helped resist movement of prototype A. It is believed that otherwise it would have slipped as in the pig test. The results of both tests suggested that the stiffness of prototype B was sufficient to hold its position without additional support. In both models, no trauma to the rectum or anus was observed after the procedure. To summarize the results of the pig and cadaver testing, the PRD was deemed successful in holding air and water in the colon, even under non-ideal or extreme conditions, in which there was no buttocks or anal tone.

The pig and cadaver test results suggest that, with the proper stiffness, prototype B of the PRD passes the key functional requirements to achieve its intended purpose. The prototype made of material having a Shore A hardness of 40 HA performed well, suggesting that slightly higher values would also achieve the desired results. Accordingly, an overall hardness range of 40 to 65 HA is believed to be acceptable. A balancing between stability, ease of handling, and friction suggests a revised target range of 55 HA to 60 HA.

The inventive colonoscopy pressure retention device (PRD) described herein provides means for retaining air or water that is pumped into the colon during a colonoscopy procedure to enhance visualization, essentially acting as an artificial sphincter. The device may be advantageously configured to be applied after the colonoscope has been already been inserted into the patient, allowing the physician to make a needed adjustment if too much pressure is being lost during the procedure without requiring retraction of the scope. The PRD is molded from a medical grade silicone material, making it cost-effective as a sanitary, disposable single-use device.

1. A pressure retention device, comprising:
a bottom flange configured to fit within a patient's intergluteal cleft in contact with an anal opening, the bottom flange have a central flange opening;
a neck portion extending from the bottom flange, the neck portion having a co-axial channel therethrough aligned with the central flange opening, the co-axial channel having an annular seal therein, the neck portion configured to extend from an exterior of the anal opening through an anal sphincter; and
an upper portion disposed at an end of the neck portion opposite the bottom flange, the upper portion having a central upper opening aligned with the co-axial channel and pair of resilient flaps extending radially therefrom, each flap having a partial channel so that, when the flaps are folded radially inward, the partial channels define an extension of the co-axial channel and the flaps form an insertion tip;
wherein the co-axial channel and the annular seal are configured to fit closely over an outer surface of an endoscope so that, when the flaps are inserted into the patient's anal opening and the neck portion is disposed substantially in alignment with the anal sphincter, the flaps at least partially relax to radially extend from the upper portion and aid in the retention of the device within a patient's anal canal.

2. The device of claim 1, wherein the flaps are connected to the upper portion by a grooved hinge, wherein the flaps fold along the hinge upon application of pressure to an outer portion of each flap.

3. The device of claim 1, wherein the bottom flange, neck portion, and upper portion are formed as a single piece of a flexible, biocompatible material.

4. The device of claim 3, further comprising a continuous longitudinal slit formed through a side of each of the bottom flange, neck portion, and the upper portion, the slit configured for accessing the co-axial channel radially for positioning of the device onto the endoscope.

5. The device of claim 4, further comprising a semi-cylindrical clip configured to slide over the neck portion to seal the slit after the device is positioned over the endoscope.

6. The device of claim 3, wherein the flexible, biocompatible material is a medical-grade silicone.

7. The device of claim 3, wherein the flexible, biocompatible material is medical grade polydimethylsiloxane (PDMS).

8. The device of claim 3, wherein the flexible, biocompatible material has a Shore A hardness within a range of 40 HA to 65 HA.

9. The device of claim 1, wherein the annular seal comprises a rib extending radially inward and angled toward the upper portion.

10. The device of claim 1, wherein the bottom flange has an oval shape, wherein the oval shape has a long axis aligned with the patient's intergluteal cleft.

11. The device of claim 10, wherein the bottom flange further comprises a ridged topography aligned with the long axis of the oval shape, the ridged topography configured to ergonomically conform to the patient's intergluteal cleft.

12. The device of claim 1, wherein each flap has a tapered lower surface so that the insertion tip has a generally conical shape when the flaps are folded inward.

13. A pressure retention device comprising:
a neck portion having a co-axial channel therethrough dimensioned to slidably receive an endoscope;
an annular seal extending radially inward within the co-axial channel to contact an outer surface of the endoscope;
a bottom flange disposed at a first end of the neck portion, the bottom flange configured to fit within a patient's intergluteal cleft in contact with an anal opening; and
an upper portion disposed at a second end of the neck portion, the upper portion having a pair of radially-extending resilient flaps that, when folded radially inward, form an insertion tip that resiles to extend radially after insertion through the anal opening, wherein each flap has a tapered lower surface so that the insertion tip has a generally conical shape when the flaps are folded inward;
wherein the annular seal generates a pressure seal to resist release of a colon pressurizing fluid through the anal opening.

14. The device of claim 13, wherein the flaps are connected to the upper portion by a grooved hinge, wherein the flaps fold along the hinge upon application of pressure to an outer portion of each flap.

15. The device of claim 13, wherein the neck portion, bottom flange, and upper portion are molded from a single piece of a flexible, biocompatible material.

16. The device of claim 15, wherein the flexible, biocompatible material is polydimethylsiloxane (PDMS).

17. The device of claim 15, wherein the flexible, biocompatible material has a Shore A hardness within a range of 40 HA to 65 HA.

18. The device of claim 15, further comprising a continuous longitudinal slit formed through a side of each of the neck portion, bottom flange, and upper portion, the slit configured for accessing the co-axial channel radially for positioning of the device onto the endoscope.

19. The device of claim 18, further comprising a semi-cylindrical clip configured to slide over the neck portion to seal the slit after the device is positioned over the endoscope.

20. The device of claim 12, wherein the annular seal comprises a rib extending radially inward and angled toward the upper portion.

21. The device of claim 12, wherein the bottom flange has an oval shape, wherein the oval shape has a long axis aligned with the patient's intergluteal cleft.

22. The device of claim 21, wherein the bottom flange further comprises a ridged topography aligned with the long axis of the oval shape, the ridged topography configured to ergonomically conform to the patient's intergluteal cleft.
